(19) United States
(12) Patent Application Publication
(10) Pub. No.: US 2022/0323037 A1
(43) Pub. Date: Oct. 13, 2022

(54) SYSTEM AND METHOD FOR UTILIZING PATIENT-SPECIFIC EMISSION-BASED BODY CONTOUR DETECTION

(71) Applicant: GE PRECISION HEALTHCARE LLC, Wauwatosa, WI (US)

(72) Inventors: Michal Maria Merman, Nesher (IL); Yariv Grobshtein, Haifa (IL)

(21) Appl. No.: 17/224,394

(22) Filed: Apr. 7, 2021

(51) Int. Cl. A61B 6/00; G06T 7/13; G06T 11/00; A61B 6/03

(52) U.S. Cl. CPC A61B 6/545; G06T 7/13; G06T 11/008; A61B 6/037; A61B 6/4266; G06T 2207/20116; G06T 2211/412

(57) ABSTRACT

An imaging system is provided that includes a gantry defining a bore configured to accept an object to be imaged, wherein the gantry is configured to rotate about the bore. The system includes multiple detector units mounted to the gantry and configured to rotate with the gantry around the bore in rotational steps, each detector unit configured to sweep about a corresponding axis and acquire imaging information while sweeping about the corresponding axis. The system includes at least one processor operably coupled to at least one of the detector units that is configured to acquire, during an initial portion of a scan, imaging information of the object based on an initial contour and to detect an actual emission contour based on the imaging information. The processor is configured to update a scan sweep plan based on the detected actual emission contour for a remaining portion of the scan.

### BACKGROUND

The subject matter disclosed herein relates to medical imaging systems and, more particularly, to radiation detection systems.

In nuclear medicine (NM) imaging, such as single photon emission computed tomography (SPECT) or positron emission tomography (PET) imaging, radiopharmaceuticals are administered internally to a patient. Detectors (e.g., gamma cameras), typically installed on a gantry, capture the radiation emitted by the radiopharmaceuticals and this information is used, by a computer, to form images. The NM images primarily show physiological function of, for example, the patient or a portion of the patient being imaged.

An NM imaging system may be configured as a multi-head imaging system having several individual detectors distributed about the gantry. Each detector (e.g., detector head) may pivot or sweep to provide a range over which the detector may acquire information that is larger than a stationary field of view of the detector. In a multi-head imaging system, each scanning detector head needs to divide the available scan time between multiple acquisition angles, where the total angular range to be covered is usually defined by a pre-defined fixed contour or by a contour acquired by some additional means (e.g., optical sensors, capacitive proximity sensors, cameras, CT scan, etc.). Most contour detection techniques rely on detecting the physical contour (e.g., body contour), which is then scanned by the detectors in acquisition sweeps to detect emission and map the physiological activity within the body. However, in many cases, the physical contour is larger, sometimes significantly, than the active emitting body contour, due to items (e.g., clothing, blankets, extending tubes, mechanical obstructions, patient table, etc.) that add to the acquired contour size. This causes some of the views to be acquired out of the range of the actual emitting body contour, resulting in projections with poor photon counts and negligible contribution to image quality. In addition, scans are longer than necessary due to the acquisition of these views out of the range of the actual emitting body contour.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a nuclear medicine multi-head imaging system is provided. The system includes a gantry defining a bore configured to accept an object to be imaged, wherein the gantry is configured to rotate about the bore. The system also includes multiple detector units mounted to the gantry and configured to rotate with the gantry around the bore in rotational steps, each detector unit configured to sweep about a corresponding axis and acquire imaging information while sweeping about the corresponding axis. The system further includes at least one processor operably coupled to at least one of the detector units. The at least one processor is configured to acquire, during an initial portion of a scan, imaging information of the object based on an initial contour. The at least one processor is also configured to detect an actual emission contour based on the imaging information. The at least one processor is also configured to update a scan sweep plan based on the detected actual emission contour for a remaining portion of the scan.

In another embodiment, a method for utilizing a nuclear medicine multi-head imaging system is provided. The method includes acquiring via multiple detector units, during an initial portion of a scan, imaging information of an object based on an initial contour. The method also includes detecting an actual emission contour based on the imaging information. The method further includes updating a scan sweep plan based on the detected actual emission contour for a remaining portion of the scan.

In a further embodiment, a non-transitory computer-readable medium is provided. The computer-readable medium includes processor-executable code that when executed by a processor causes the processor to perform actions. The actions include acquiring via multiple detector units of a nuclear medicine multi-head imaging system, during an initial portion of a scan, imaging information of an object based on an initial contour. The actions also include detecting an actual emission contour based on the imaging information. The actions further include updating a scan sweep plan based on the detected actual emission contour for a remaining portion of the scan.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a schematic view of an embodiment of a nuclear imaging system, in accordance with aspects of the disclosed techniques;

FIG. 2 is a flow chart of an embodiment of a method for utilizing patient-specific emission-based body contour detection in medical imaging, in accordance with aspects of the disclosed techniques;

FIG. 3 is a schematic diagram of an embodiment of a workflow for utilizing patient-specific emission-body contour detection in medical imaging, in accordance with aspects of the disclosed techniques;

FIG. 4 is a schematic diagram of an embodiment of an alternative workflow for utilizing patient-specific emission-body contour detection in medical imaging, in accordance with aspects of the disclosed techniques;

FIG. 5 is a schematic diagram of illustrating the benefit of utilizing patient-specific emission-body contour detection in a whole-body or multi-field of view imaging application, in accordance with aspects of the disclosed techniques;

FIG. 6 is a collection of images depicting the utilization of patient-specific emission-body contour detection (e.g., for feet), in accordance with aspects of the disclosed techniques; and

FIG. 7 is a collection of images depicting the utilization of patient-specific emission-body contour detection (e.g., for a torso region), in accordance with aspects of the disclosed techniques.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

The present disclosure provides systems and methods for utilizing patient-specific emission-based body contour detection in medical imaging. Detecting a true emission contour enables automatic scan range optimization and improves scan and reconstruction time economy. During a scan with a nuclear medicine (NM) multi-head imaging system (e.g., for SPECT imaging), imaging information or data (e.g., photon counts) may be acquired of an object, during an initial portion of the scan, based on an initial contour (e.g., physical or body or proximity contour). The initial contour may be larger than an actual emission contour from the object due to items (e.g., clothing, blankets, extending tubes, mechanical obstructions, patient table, etc.) that add to the contour size. The imaging information acquired during the initial portion of the scan may be utilized to detect an actual emission contour of the object. Based on the detected actual emission contour, a scan sweep plan is updated (e.g., sweep range, scan range, scan time, movement plan, etc.) for a remaining portion of the scan. In the disclosed embodiments, the detection of the actual emission contour may only utilize an emission window (as opposed to both the emission and scatter windows) as the energy set. Data (e.g., partial data) from an initial portion of the scan (e.g., diagnostic scan) may be utilized along the data acquired from the rest of the scan so that all acquired data is utilized in the reconstruction of images, thus, avoiding any photon loss. In certain embodiments, the scan sweep plan may be continuously updated as the actual emission contour is updated as more subsequent imaging information is acquired. In a whole-body workflow or multi-field of view (FOV) scans, the detection of an actual emission contour and updating of a scan sweep plan may be conducted for scans for different positions (and FOVS) of an object along an imaging axis (e.g., from head to feet).

The disclosed embodiments, with regard to scan planning, may result in a reduction in scan time (e.g., due to not scanning empty angles or acquiring imaging information outside of the actual emitting contour) without compromising image quality, thus, enabling a higher throughput. In addition, the disclosed embodiments may increase image quality in the same acquisition time. Also, the disclosed embodiments may enable utilizing a reduced patient dose while maintaining the image quality and scan time. Further, the disclosed embodiments, enable an automated whole body multi-FOV workflow to be utilized without added preview steps or user marking of true body contours. The disclosed embodiments, with regard to reconstruction, may result in photon statistics gain and better image quality. In addition, the disclosed embodiments, may enable a shorter reconstruction time and faster convergence due to a smaller image size. Further, the disclosed embodiments, by providing faster reconstruction, may enable the utilization of less expensive hardware. Still further, the disclosed embodiments, may enable more precise quantitative results due to correctly defining the image boundaries.

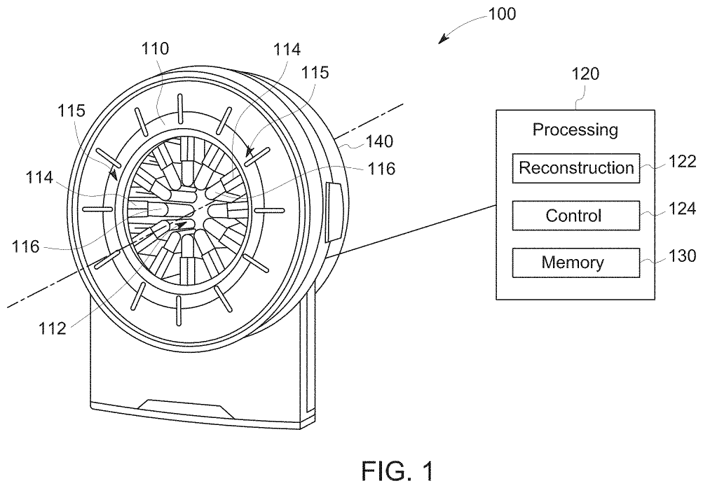

FIG. 1 provides a schematic view of a nuclear medicine (NM) multi-head imaging system 100 in accordance with various embodiments. Generally, the imaging system 100 is configured to acquire imaging information or data (e.g., photon counts) from an object to be imaged (e.g., a human patient) that has been administered a radiopharmaceutical. The depicted imaging system 100 includes a gantry 110 and a processing unit 120.

The gantry 110 defines a bore 112. The bore 112 is configured to accept an object to be imaged (e.g., a human patient or portion thereof). As seen in FIG. 1, a plurality of detector units 115 are mounted to the gantry 110. In the illustrated embodiment, each detector unit 115 includes an arm 114 and a head 116. The arm 114 is configured to articulate the head 116 radially toward and/or away from a center of the bore 112 (and/or in other directions), and the head 116 includes at least one detector, with the head 116 disposed at a radially inward end of the arm 114 and configured to pivot to provide a range of positions from which imaging information is acquired.

The detector of the head 116, for example, may be a semiconductor detector. For example, a semiconductor detector in various embodiments may be constructed using different materials, such as semiconductor materials, including Cadmium Zinc Telluride (CdZnTe), often referred to as CZT, Cadmium Telluride (CdTe), and Silicon (Si), among others. The detector may be configured for use with, for example, nuclear medicine (NM) imaging systems, positron emission tomography (PET) imaging systems, and/or single photon emission computed tomography (SPECT) imaging systems.

In various embodiments, the detector may include an array of pixelated anodes, and may generate different signals depending on the location of where a photon is absorbed in the volume of the detector under a surface of the detector. The absorption of photons from certain voxels corresponding to particular pixelated anodes results in charges generated that may be counted. The counts may be correlated to particular locations and used to reconstruct an image.

In various embodiments, each detector unit 115 may have a corresponding stationary field of view (FOV) that is oriented toward the center of the bore 112. Furthermore, each detector unit 115 in the illustrated embodiment is configured to acquire imaging information over a sweep range of the given detector unit 115. Thus, each detector unit 115 may collect information over a range larger than a field of view defined by a stationary detector unit. It may be noted that, generally, the sweeping range over which a detector unit 115 may potentially pivot may be larger than the corresponding FOV during acquisition. In some cameras, the sweeping range that a detector may pivot may be unlimited (e.g., the detector may pivot a full 360 degrees), while in some embodiments the sweeping range of a detector may be constrained, for example over 180 degrees (from a −90 degree position to a +90 degree position relative to a position oriented toward the center of the bore). The gantry 110 may be rotatable to different positions, with the detector units 115 rotating with the gantry 110. For example, with the gantry 110 in a first position, the individual detector units 115 may be swept to acquire a first set or amount of imaging information. Then, the gantry 110 may be moved to a second position (e.g., rotated to a new position, with the detector units 115 moving or rotating with the gantry 110). With the gantry 110 in the second position, the individual detector units 115 may be swept again to acquire a second set or amount of imaging information.

In some embodiments, the system 100 further includes a CT (computed tomography) detection unit 140. The CT detection unit 140 may be centered about the bore 112. Images acquired using both NM and CT by the system are accordingly naturally registered by the fact that the NM and CT detection units are positioned relative to each other in a known relationship. A patient may be imaged using both CT and NM modalities at the same imaging session, while remaining on the same bed, which may transport the patient along the common NM-CT bore 112.

With continued reference to FIG. 1, the depicted processing unit 120 is configured to acquire imaging information or data (e.g., photon counts) via the detector units 115. The imaging information is acquired within a contour boundary. In certain embodiments, the contour boundary may be a physical contour (e.g., body or proximity contour) that is larger, sometimes significantly, than the active emitting body contour, due to items (e.g., clothing, blankets, extending tubes, mechanical obstructions, patient table, etc.) that add to the acquired contour size. This causes some of the views to be acquired out of the range of the actual emitting body contour, resulting in projections with poor photon counts and negligible contribution to image quality. In certain embodiments, as described in greater detail below, the contour boundary may be the actual emission contour as detected from the acquired imaging information.

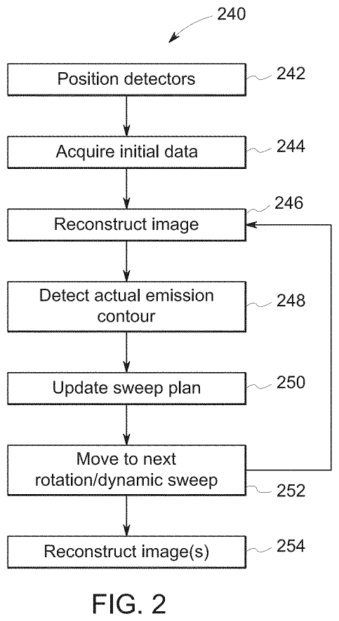

FIG. 2 is a flow chart of an embodiment of a method 240 for utilizing patient-specific emission-based body contour detection in medical imaging. One or more steps of the method 240 may be performed by one or more components of the NM multi-head imaging system 100 in FIG. 1 (e.g., processing unit 120). It should be noted that the imaging described below is conducted after the introduction of a radiopharmaceutical into the object (e.g., patient) to be imaged. The method 240 includes positioning the detectors (e.g., of the system 100) into their expected locations about the object (e.g., patient) to be imaged to start an acquisition of data (e.g., imaging information) (block 242). In certain embodiments, the expected locations may be based on a predefined location. In other embodiments, the expected location may be based on an initial contour (e.g., proximity or physical or body contour). In other embodiments, the initial contour may be based on the output of mechanical positions sensors (e.g., capacitive proximity sensors). In certain embodiments, the initial contour (e.g., optical contour) may be determined optically (e.g., via optical sensors, cameras, etc.). In other embodiments, the initial contour (e.g., CT contour) may be determined based on a CT scan. In certain embodiments, other means of proximity detection of the system (besides those mentioned above) may be utilized to define the initial contour.

The method 240 also includes acquiring initial data (e.g., imaging information) via the system (e.g., system 100 in FIG. 1) (block 244). In certain embodiments, the initial data is acquired from a preview scan. In certain embodiments, the initial data may be acquired during an initial portion of a planned scan. For example, the initial data may be acquired during a first rotation or a first dynamic sweep of a planned scan. In other embodiments, the initial data may be acquired during a first rotation within a predefined fixed sweep range or one or more predefined dynamic sweeps.

The method 240 further includes reconstructing an image (e.g., an initial image or emission image) from the initial data (block 246). In certain embodiments, the initial data may be partial scan data (e.g., acquired from a first rotation out of multiple rotations, first few dynamic sweeps, preview scan, etc.). Prior to reconstruction, the data may be preprocessed. For example, preprocessing of projections (e.g., sums, filtering, thresholding, etc.) may occur. The method 240 even further includes detecting an actual or true emission contour of the object within the image (block 248). In particular, segmentation may be utilized to detect the actual emission contours. For example, segmentation may occur via threshold/histogram operations. Segmentation may also occur via masking, filters, and/or morphological operations. Segmentation may also occur via contour growing methods or other image segmentation techniques (e.g., a pre-trained neural network for segmentation).

The method 240 even further includes updating a sweep or scan plan based on the detected actual emission contour (block 250) (e.g., for subsequent rotations/dynamic sweeps). Updating the sweep plan may include recalculating a motion plan utilizing the actual emission contour. For example, the angular range of a sweep or a motion of the detectors (e.g., cameras) may be recalculated. If a view in the original plan does not fall within the actual emission contour, the view may be eliminated, thus, only the emitting range is swept. In certain embodiments, the sweep plan for subsequent rotations/dynamic sweeps may be calculated from scratch. In certain embodiments, updating the sweep plan may include reevaluating a scan time based on the actual emission contour. Reevaluation may include retaining an original scan time resulting in more photons per view and a gain in counts. Reevaluation may also include retaining the planned time per view and calculating how much time is gained by not scanning eliminated views. In certain embodiments, updating the sweep plan may include reevaluating or updating expected scan counts and estimating how much time is needed to reach a stop-on-counts criterion or threshold from which the scan times may be updated accordingly.

The method 240 still further includes moving to the next rotation or dynamic sweep following the updated scan plan or sweep plan (block 252). In certain embodiments, the remaining portion of the scan may be conducted according to the updated scan plan or sweep plan. In other embodiments, the sweep or scan plan may be continuously updated over the remaining portion of the scan (e.g., as more imaging information or data is acquired after one or more subsequent rotations or sweeps). The continuous update may occur after a pre-defined number of rotations or sweeps (e.g., 1, 2, 3, etc.). In continuously updating the sweep plan (and the actual emission contour), after each rotation or predefined number of rotations, the steps from block 246 to block 252 may be repeated. In continuously updating the actual emission contour, all of the data up until that point (including the data from the initial portion of the scan) may be utilized in reconstructing the image for detecting the actual emission contour.

The method 240 yet further includes reconstructing one or more images from the scan upon completion of the scan (block 254). The one or more images are reconstructed from all of the acquired data (including the data from the initial portion of the scan), thus, no counts are lost.

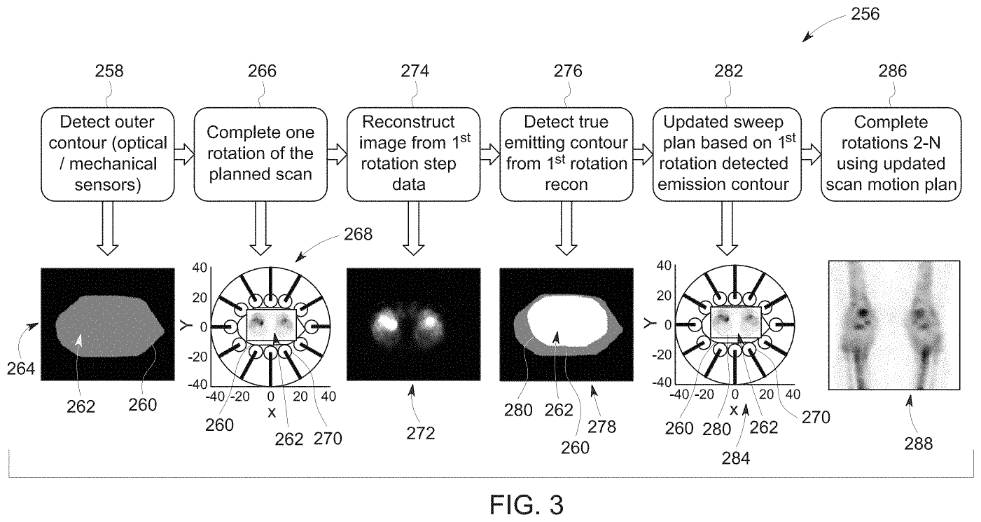

FIG. 3 is a schematic diagram of an embodiment of a workflow 256 for utilizing patient-specific emission-body contour detection in medical imaging (e.g., utilizing a NM multi-head imaging system). The workflow 256 includes detecting an outer contour (e.g., initial contour such as a physical or body contour) (block 258). The outer contour may be detected mechanically, optically, via CT, or other means as described above. An example of a detected outer contour 260 of an object 262 (e.g., patient) is shown in image 264. The workflow 256 then proceeds to completing a single rotation of a planned scan (e.g., of N rotations with N typically ranging between 4 and 6) to acquire data (e.g., image information) utilizing the initial contour (block 266). As depicted in image 268, each detector head 270 is positioned about the object 262 based on the outer contour 260. After acquisition of the initial data, the workflow 256 includes reconstructing an initial image 272 (e.g., emission image) from the data acquired during the first rotation (block 274). The workflow 256 includes detecting the true or actual emission contour from the image 272 (block 276). Image 278 depicts an actual emission contour 280 within the outer contour 260 of the object 262. Based on the detected actual emission contour 280, the workflow 256 includes updating a sweep or scan plan or scan motion plan (block 282). As depicted in image 284, the detector heads 270 acquire data via sweeps of only angles that will collect data from within the emitting region (as defined by the actual emission contour 280). Finally, the workflow 256 includes completing the remaining rotations (e.g., rotations 2-N) utilizing the updated plan from which a reconstructed image may be generated (e.g., reconstructed image 288) (block 286). In some embodiments, the actions within blocks 274 to 286 can be repeated several times (e.g., after completion of each N rotation step) to optimize acquisition of emission data for the final reconstructed image 288.

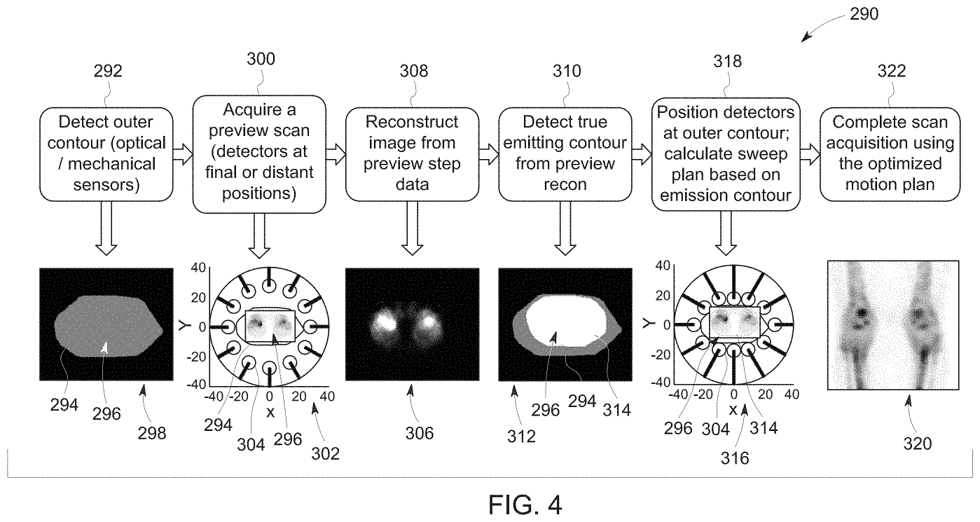

FIG. 4 is a schematic diagram of an embodiment of an alternative workflow 290 for utilizing patient-specific emission-body contour detection in medical imaging (e.g., utilizing a NM multi-head imaging system). The workflow 290 includes detecting an outer contour (e.g., initial contour such as a physical or body contour) (block 292). The outer contour may be detected mechanically, optically, via CT, or other means as described above. An example of a detected outer contour 294 of an object 296 (e.g., patient) is shown in image 298. The workflow 290 then proceeds with acquiring a preview scan (block 300). As depicted in image 302, each detector head 304 is positioned at distant position from the object 296 and the outer contour 294. After acquisition of the preview scan, the workflow 290 includes reconstructing an initial image (e.g., emission image) 306 from the data acquired during the preview scan (block 308). The workflow 290 includes detecting the true or actual emission contour from the image 306 (block 310). Image 312 depicts an actual emission contour 314 within the outer contour 294 of the object 296. Based on the detected actual emission contour 314, the workflow 290 includes positioning the detectors at the outer contour 294 (as indicated in image 316) calculating a sweep or scan plan or scan motion plan based on the actual emission contour 314 (block 318). As depicted in image 316, the detector heads 304 acquire data via sweeps of only angles that will collect data from within the emitting region (as defined by the actual emission contour 314). Finally, the workflow 290 includes completing the scan utilizing the calculated plan from which a reconstructed image may be generated (e.g., reconstructed image 320) (block 322).

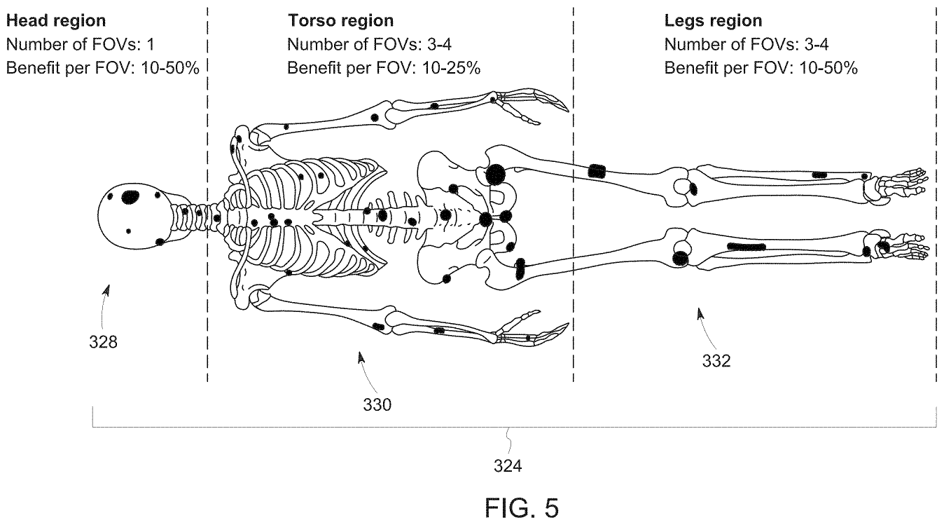

The method 240 and workflows 256, 290 described above may be adapted to enable automatic organ detection for focused scans on multi-head systems and allow improved workflow by reducing the need for user focus region of interest marking. In addition, these techniques may be utilized in an application utilizing a whole-body workflow or multi-FOV scans. Typically, in a whole-body workflow or multi-FOV scans application, a significant amount of total time is wasted on regions where there is no activity or emission (e.g., due to accumulation of time wasted in each FOV). In particular, utilizing other methods 0 is not feasible for multi-FOV scans (manual marking) or would waste significant time while not providing the necessary information (e.g., CT, mechanical or optical contour detection, etc.). The techniques described above (e.g., utilizing patient-specific emission-body contour detection) may be applied automatically on each FOV (e.g., different position) resulting in a scan time/photon counts gain per each FOV and a high total gain for the entire scan. In other words, the actual emission contour may be detected and utilized for determining or updating a sweep plan for each FOV. As depicted in FIG. 5, a whole-body workflow or multi-FOV scans application may be conducted along an imaging axis 324 of an object 326 (e.g., patient) from head to toes. The object 326 includes multiple regions (e.g., head region 328, torso region 330, legs regions 332) at different positions along the imaging axis 324. Each region may be associated with a different number of FOVS. For example, the head region 328, torso region 330, and the legs region 332 may include FOVS of 1, 3 to 4, and 3 to 4, respectively. The estimated benefit (defined as expected scan time/photon counts as a percentage) for each FOV for each region 328, 330, 332 is approximately 10 to 50 percent, 10 to 25 percent, and 10 to 50 percent, respectively.

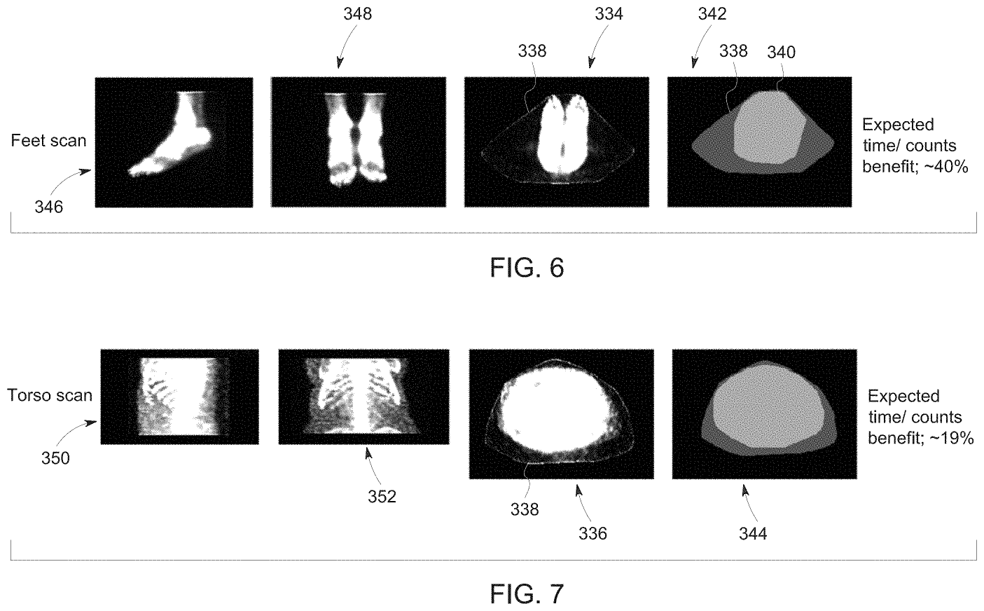

FIGS. 6 and 7 depict the utilization of patient-specific emission-body contour detection techniques described above for the feet and the torso region, respectively. Images 334, 336 are initial images (e.g., emission images) based on imaging data acquired from a single rotation (e.g., utilizing a proximity contour 338) during a scan of the feet and the torso region, respectively. From the initial images 334, 336, the actual emission contour 340 can be detected as indicated in images 342, 344 for the feet and the torso region, respectively. Reconstructed images 346, 348 for the feet and reconstructed images 350, 352 are reconstructed from imaging data acquired from the subsequent rotations utilizing the actual emission contour 340 (as well as the imaging data acquired during the first rotation). Based on a study of the acquisition of data from the first rotation of 41 scans, a benefit (defined as expected scan time/photon counts as a percentage) of approximately 40 and approximately 19 percent may be achieved for scanning the feet and the torso region, respectively.

Technical effects of the disclosed embodiments include utilizing patient-specific emission-based body contour detection in medical imaging. With regard to scan planning, the disclosed embodiments may result in a reduction in scan time without compromising image quality, thus, enabling a higher throughput. In addition, the disclosed embodiments may increase image quality in a same acquisition time. Also, the disclosed embodiments may reduce patient dose while maintaining image quality and scan time. Further, the disclosed embodiments, enable an automated whole body multi-FOV workflow to be utilized without added preview steps or user marking of true body contours. The disclosed embodiments, with regard to reconstruction, may result in photon statistics gain and better image quality. In addition, the disclosed embodiments, may enable a shorter reconstruction time and faster convergence due to a smaller image size. Further, the disclosed embodiments, by providing faster reconstruction, may enable the utilization of less expensive hardware. Still further, the disclosed embodiments, may enable more precise quantitative results due to correctly defining the image boundaries.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function]. . . ” or “step for [perform]ing [a function]. . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A nuclear medicine multi-head imaging system, comprising:
a gantry defining a bore configured to accept an object to be imaged, wherein the gantry is configured to rotate about the bore;
a plurality of detector units mounted to the gantry and configured to rotate with the gantry around the bore in rotational steps, each detector unit configured to sweep about a corresponding axis and acquire imaging information while sweeping about the corresponding axis; and
at least one processor operably coupled to at least one of the detector units, wherein the at least one processor is configured to:
acquire, during an initial portion of a scan, imaging information of the object based on an initial contour;
detect an actual emission contour based on the imaging information; and
update a scan sweep plan based on the detected actual emission contour for a remaining portion of the scan.

2. The system of claim 1, wherein the at least one processor is configured to acquire subsequent imaging information of the object based on the detected actual emission contour during the remaining portion of the scan.

3. The system of claim 2, wherein the at least one processor is configured to reconstruct one or more images utilizing both the imaging information acquired during the initial portion of the scan and subsequent imaging information acquired during the remaining portion of the scan.

4. The system of claim 1, wherein the initial contour is determined based on a computed tomography scan, data acquired optically or mechanically, or a predefined location.

5. The system of claim 1, wherein the at least one processor is configured to detect the actual emission contour utilizing only an emission window within the imaging information.

6. The system of claim 1, wherein the at least one processor is configured to continuously update the scan sweep plan as more subsequent imaging information is acquired.

7. The system of claim 1, wherein the at least one processor is configured to reconstruct an initial image based on the imaging information acquired during the initial portion of the scan and to segment the initial image to detect actual emission contour.

8. The system of claim 1, wherein the initial portion of the scan comprises a first planned rotation or a portion of the first planned rotation utilizing the initial contour, a first rotation or a portion of the first rotation within a predefined fixed sweep range, or one or more initial predefined dynamic sweeps.

9. The system of claim 1, wherein updating the scan sweep plan comprises updating a sweep range, a scan range, or a scan time.

10. The system of claim 1, wherein the at least one processor is configured to conduct a respective scan for different positions of the object along an imaging axis, each different position associated with a respective field of view, and wherein the at least one processor is configured to:
acquire, during a respective initial portion of each respective scan for each different position, respective imaging information of the object based on a respective initial contour;
detect a respective actual emission contour based on the respective imaging information for each respective scan; and
update a respective scan sweep plan based on the respective detected actual emission contour for a respective remaining portion of each respective scan.

11. A method for utilizing a nuclear medicine multi-head imaging system, comprising:
acquiring via a plurality of detector units, during an initial portion of a scan, imaging information of an object based on an initial contour;
detecting an actual emission contour based on the imaging information; and
updating a scan sweep plan based on the detected actual emission contour for a remaining portion of the scan.

12. The method of claim 11, comprising acquiring subsequent imaging information of the object based on the detected actual emission contour during the remaining portion of the scan.

13. The method of claim 12, comprising reconstructing one or more images utilizing both the imaging information acquired during the initial portion of the scan and subsequent imaging information acquired during the remaining portion of the scan.

14. The method of claim 11, wherein detecting the actual emission contour comprises utilizing only an emission window within the imaging information.

15. The method of claim 11, comprising continuously updating the scan sweep plan as more subsequent imaging information is acquired.

16. The method of claim 11, comprising conducting a respective scan for different positions of the object along an imaging axis, each different position associated with a respective field of view, acquiring, during a respective initial portion of each respective scan for each different position, respective imaging information of the object based on a respective initial contour, detecting a respective actual emission contour based on the respective imaging information for each respective scan, and updating a respective scan sweep plan based on the respective detected actual emission contour for a respective remaining portion of each respective scan.

17. A non-transitory computer-readable medium, the computer-readable medium comprising processor-executable code that when executed by a processor, causes the processor to:
acquire via a plurality of detector units of a nuclear medicine multi-head imaging system, during an initial portion of a scan, imaging information of an object based on an initial contour;
detect an actual emission contour based on the imaging information; and
update a scan sweep plan based on the detected actual emission contour for a remaining portion of the scan.

18. The non-transitory computer-readable medium of claim 17, wherein the processor-executable code when executed by the processor, causes the processor to acquire subsequent imaging information of the object based on the detected actual emission contour during the remaining portion of the scan and to reconstruct one or more images utilizing both the imaging information acquired during the initial portion of the scan and subsequent imaging information acquired during the remaining portion of the scan.

19. The non-transitory computer-readable medium of claim 17, wherein the processor-executable code when executed by the processor, causes the processor to detect the actual emission contour utilizing only an emission window within the imaging information.

20. The non-transitory computer-readable medium of claim 17, wherein the processor-executable code when executed by the processor, causes the processor to continuously update the scan sweep plan as more subsequent imaging information is acquired.
